(19) United States
(12) Plant Patent Application Publication
(10) Pub. No.: US 2015/0047083 P1
(43) Pub. Date: Feb. 12, 2015

(54) MANDARIN TREE NAMED 'C4-15-19'

(50) ;

(71) Applicant: Florida Foundation Seed Producers, Inc., Marianna, FL (US)

(72) Inventor: Jude W. Grosser, Winter Haven, FL (US)

(73) Assignee: Florida Foundation Seed Producers, Inc., Marianna, FL (US)

(21) Appl. No.: 13/987,563

(22) Filed: Aug. 7, 2013

(51) Int. Cl. A01H 5/00

(52) U.S. Cl. PLT/202

(57) ABSTRACT

An interploid cross (conventional breeding) of SugarBelle® ‘LB8-9-9’ mandarin hybrid with a somatic hybrid of [Nova mandarin hybrid+Succari sweet orange], followed by embryo rescue, led to the recovery of the seedless triploid hybrid known as ‘C4-15-19’. This is the first triploid citrus hybrid released by the University of Florida that was fathered by a somatic hybrid produced years before via protoplast fusion. Trees of ‘C4-15-19’ are productive, producing an attractive peelable tangerine fruit with robust sweet flavor, maturing in October/November under Florida conditions. Fruit size is small-to-medium, and quite uniform.

### LATIN NAME OF THE GENUS AND SPECIES OF THE PLANT CLAIMED

Citrus reticulata

### VARIETY DENOMINATION

‘C4-15-19’

### BACKGROUND OF THE INVENTION

The present invention relates to a new and distinct variety of mandarin tree named ‘C4-15-19’. ‘C4-15-19’ is a triploid hybrid from an interploid cross of diploid monoembryonic ‘LB8-9’ (U.S. Plant Pat. No. 21,356) tangelo (female parent) crossed with an allotetraploid somatic hybrid of Nova mandarin hybrid (Clementine×Orlando)+Succari sweet orange (Citrus sinensis L. Osbeck) (male parent), obtained via embryo rescue. Two original trees exist at the Citrus Research and Education Center (CREC), Block 2, in Lake Alfred, Fla., one of which is grafted to ‘Carrizo’ citrus rootstock, and the other of which is grafted to a somatic hybrid of sour orange+Flying Dragon. ‘C4-15-19’ was first asexually reproduced in Lake Alfred, Fla. Trueness-to-type through asexual propagation was demonstrated by topworking (grafting) onto the somatic hybrid rootstock sour orange+Carrizo, also located in CREC Block 2.

### SUMMARY OF THE INVENTION

‘C4-15-19’ produces a ‘Clementine’-sized, seedless, peelable tangerine-type fruit with high sugar content and a very sweet flavor. Fruit matures between October and November, depending on environmental factors. Fruit pigments well under typical Florida weather conditions (without cold-induction), both internally and externally. Fruit of ‘C4-15-19’ should be clipped to avoid plugging, particularly with fruit harvested during the latter part of the harvest season. Initiation of peel removal is easy, and the top half of the fruit is generally much easier to peel than the bottom half. Peelability of ‘C4-15-19’ seems to improve during storage. The rind contains large oil glands, and pleasantly fragrant oil can be released during peeling, especially with recently harvested fruit. This characteristic is expected to diminish as the trees grow through juvenility. Although no formal yield data is available at this time, existing trees have yielded quite well, regardless of rootstock.

### BRIEF DESCRIPTION OF THE DRAWINGS

‘C4-15-19’ is illustrated by the accompanying photographs, which show the tree's from, foliage and fruit. The colors shown are as true as can be reasonably obtained by conventional photographic procedures. The photographs are of a single tree approximately 6 years old. FIGS. 1, 2, and 3 were taken in the fall of 2012. FIGS. 4, 5, 6, and 7 were taken during the early winter from the same tree.

FIG. 1—Shows a close-up of the mature fruit with the rind and cross-sectional view.

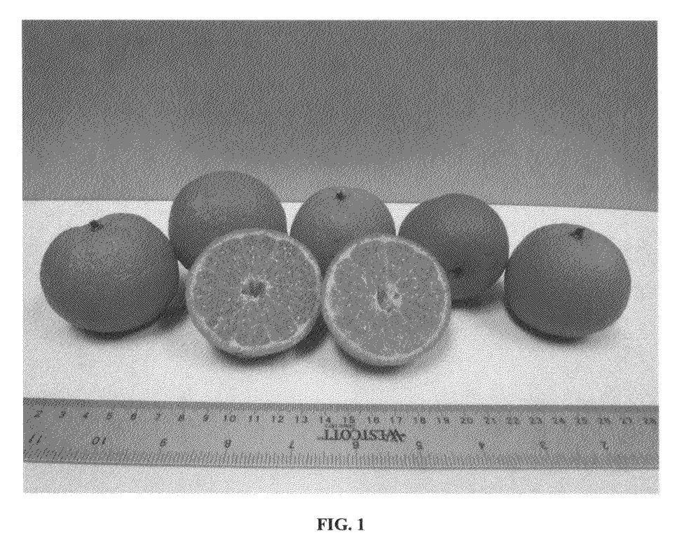

FIG. 2—Shows nearly mature fruits hanging on the tree.

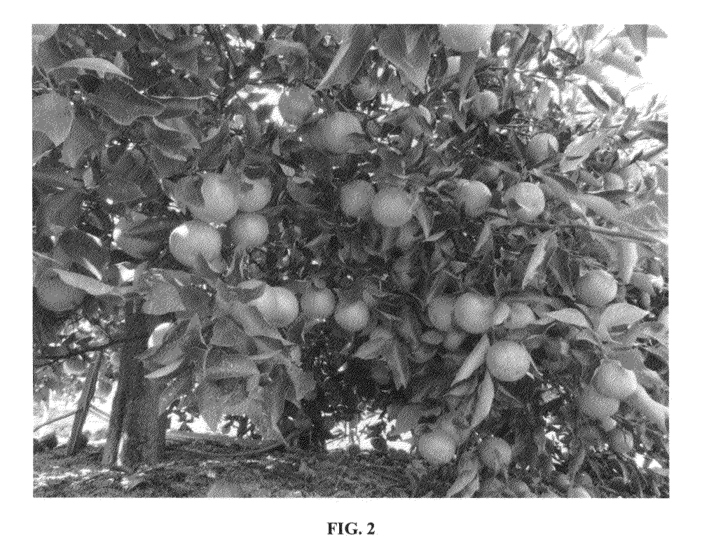

FIG. 3—Shows a close-up of leaves and nearly mature fruits.

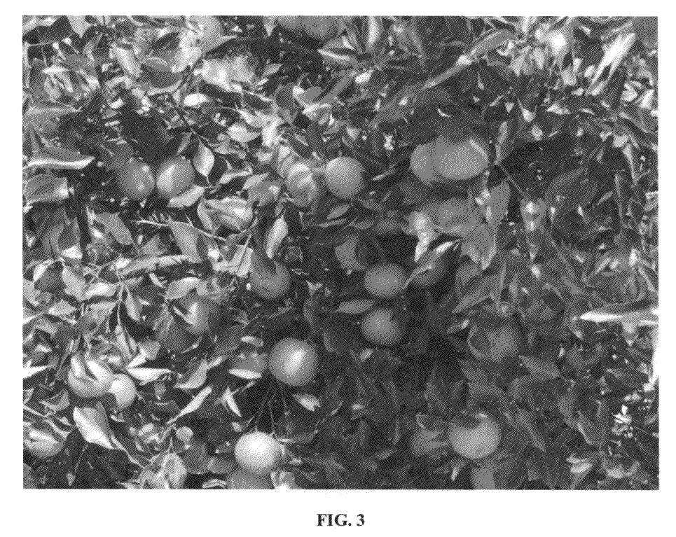

FIG. 4—Shows a close-up of the mature fruit.

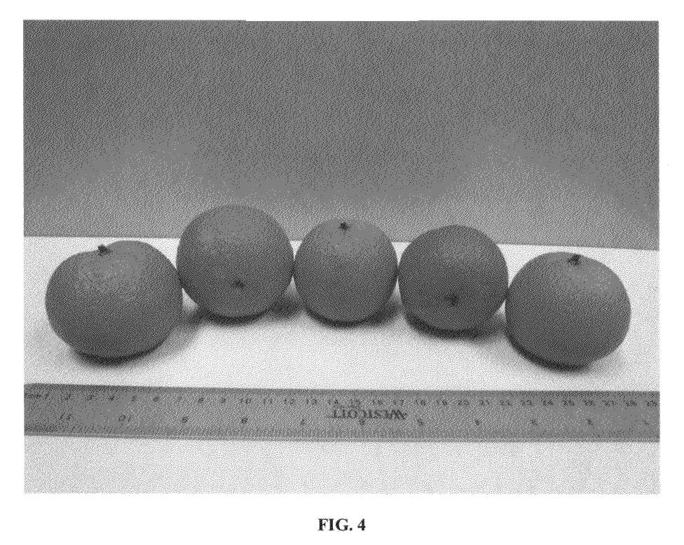

FIG. 5—Shows the overall mature plant growth habit in late summer.

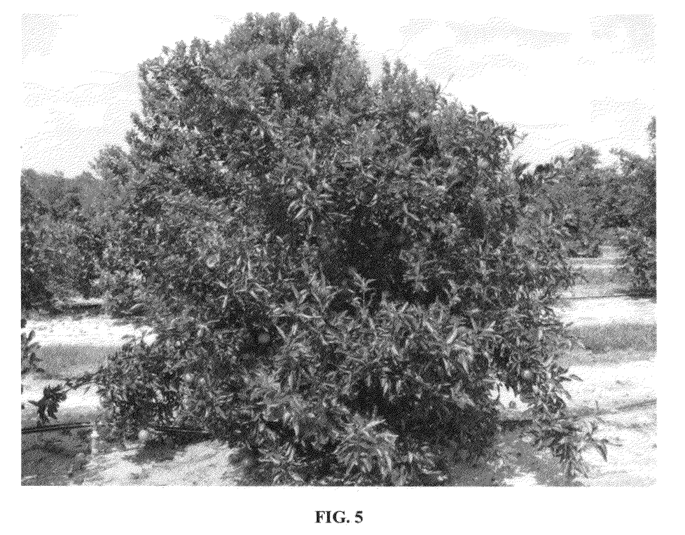

FIG. 6—Shows an additional close-up of the very mature fruit hanging on the tree.

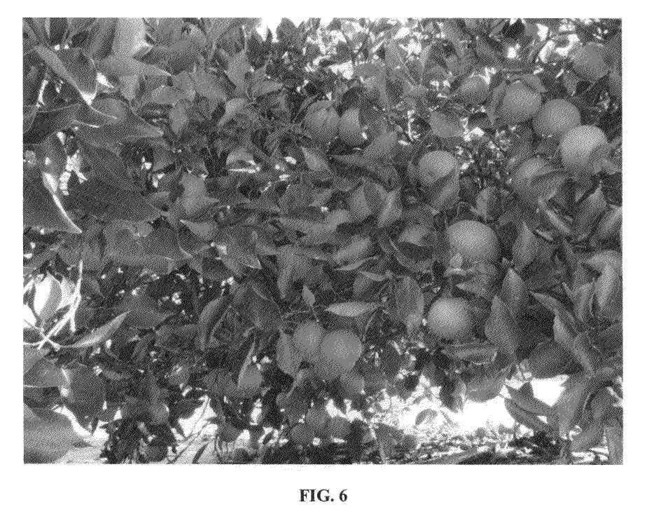

FIG. 7—Shows view of mature fruit from top, bottom, side, and center-cut angles (photo taken in November 2012).

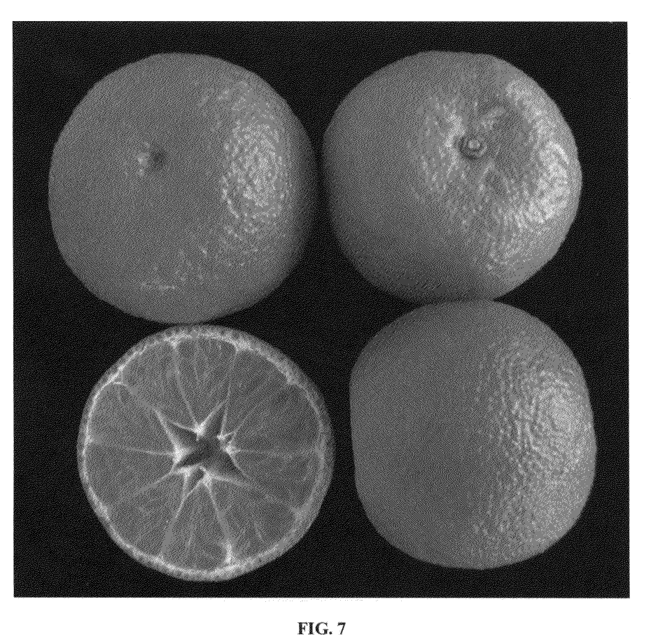

### DETAILED BOTANICAL DESCRIPTION

### Phenotypic description of Citrus reticulata ‘C4-15-19’

Classification:
   Botanical.—Citrus reticulatahybrid×somatic hybrid of C. reticulata hybrid+C. sinensis.
   Common name.—Mandarin hybrid or Tangerine.
Parentage:
   Female parent.—‘LB8-9’ tangelo (Clementine×Minneola).
   Male parent.—Somatic hybrid of ‘Nova’ mandarin hybrid (Clementine×Orlando) +‘Succari’ Sweet Orange (unpatented).
Tree:
   Ploidy.—Triploid.
   Size.—Small.
   Height.—2.2 m.
   Tree spread.—2.9 to 3.0 m.
   Vigor.—Moderately vigorous.
   Density.—Canopies are quite dense.
   Form.—The tree has a rounded shape with many lateral and upright branches growing toward low angles. Branches with fruit exhibit drooping.
   Growth habit.—Both upright and lateral growth with drooping branches.
Trunk:
   Trunk diameter.—7.4 cm at 30 cm height above the ground.
   Trunk texture.—Smooth.
   Trunk bark color.—RHS N200A (brown); irregularly striated with RHS 198A (greyed-green).
Branches:
   Crotch angle.—First crotch from 110-to 115-degree angle, middle crotch formed 65-degree angle.
   Branch length.—Branch reaches 2.7 m from the first crotch to the tip of the branch.
   Branch texture.—Relatively smooth, occasionally with small thorns or spines.
   Branch color (shoots from previous flush, hardened and 4 to 5 mm in diameter).—RHS 138A (green).
Leaves:
   Size (lamina average).—Length: 93.8 mm Width: 57.4 mm L/W ratio: 1.63.
   Thickness.—Slightly thicker than regular and average when compare to commercial mandarin hybrids.
   Type.—Simple.
   Shape.—Elliptical.
   Apex.—Retuse.
   Base.—Acute to sub-obtuse.
   Margin.—Entire and slightly undulate.
   Surface.—Upper surface: Glabrous Lower surface: Medium veins that are pinnately netted.
   Color.—Upper surface (adaxial): RHS N137B (green) Lower surface (abaxial): RHS138A (green).
   Petiole.—Shape: Brevipetiolate (shorter than leaf lamina); junction between petiole and lamina is articulate. Width (petiole wing): Narrow Shape (petiole wing): Obovate Length: 16 to 19 mm Width: 4 to 5.5 mm Color: RHS N137B (green).
Flowers and flower buds:
   Type.—Hermaphrodite.
   Bearing.—Flowers grow from leaf axillaries and leaf terminals singly and in small clusters, with most single flowers growing from leaf terminals; each flower branch consists of 2-20 flowers.
   Flower diameter.—Fully open flower with average diameter of 32 to 33 mm.
   Flower depth.—Typical flower with average depth of 15.2 mm.
   Flower blooming period.—First bloom: Observed Feb. 10, 2013 Full bloom: Observed Feb. 20, 2013.
   Flower bud size.—Length: Initial visible flower bud is 1.2 mm in length; mature flower bud is 11 mm in length. Diameter: Initial visible flower bud is 4 mm in diameter; mature flower bud is 7.2 mm in diameter. Shape: Initial visible flower bud has round ball shape; mature flower bud has elongated olive shape. Color: RHS 142C (green) for initial visible flower bud; RHS 155C (white) for mature flower bud with RHS 1C (green-yellow) spots distributed at tip of the flower bud.
   Flower petals.—Shape: Flat, spatula shaped Apex shape: Smooth, acute shaped Base shape: Even obtuse.
   Color.—Upper surface: RHS 155C (white) Lower surface: RHS 155C (white) with RHS 1C (green-yellow) spots distributed toward to the petal apex Margin: Smooth.
   Sepal.—Number: 5 per flower Shape: Delta shaped with acute angle at apex Length: 2.5 mm Width: 2.6 mm Apex shape: Triangle shaped Margin: Smooth.
   Color.—Upper surface: RHS 145C (yellow-green) Lower surface: RHS 145C (yellow-green).
   Fragrance.—Fragrant.
   Pedicel.—Length: 3.5 to 3.8 mm Diameter: 1.5 to 1.8 mm Color: RHS 145A (yellow-green).
   Reproductive organs.—Fertility: Appears self-fertile Stamen length: 11.8 to 12 mm Anther length: 3.2 mm Anther Width: 1.0 to 1.2 mm Anther color: RHS 12C (yellow) Anther filament length: 9.5 to 9.6 mm Pollen amount: Abundant.
Fruit:
   Size.—Uniform.
   Tall.—53.5 to 57.2 mm on average.
   Width.—61 to 63.5 mm on average.
   Average weight (per individual fruit).—108 g.
   Shape.—Round.
   Shape (cross-section).—Round.
   Apex.—Truncated.
   Apex cavity diameter.—N/A.
   Base.—Not necked.
   Base cavity diameter.—4.5 to 4.8 mm.
   Harvesting.—First harvest is usually around the 1st of October (based on season and rootstock); last harvest around is generally from mid-late November.
   Fruit stem (short stem connecting the fruit).—Length: 5.0 mm Diameter: 2.9 mm Color: RHS 138A (green) with RHS 197C (greyed-orange) strip.
Skin:
   Adherence.—Adherence between albedo (mesocarp) and flesh (endocarp) is medium to weak, stronger adherence at basal end of fruit, easier to peel than ‘Murcott’ tangerine, with less adherence than its female parent ‘Sugar Belle’ (patented).
   Thickness.—2.8 to 3.0 mm on average.
   Texture.—Smooth.
   Color.—Flavedo (epicarp): Ranges between RHS 21C (yellow-orange) to RHS 23C (yellow-orange) in October; RHS 24A (orange) to RHS 25N (reddish-orange) in November. Albedo (mesocarp): RHS 20C (yellow-orange).
   Stylar end.—Closed.
   Rind oil cell density.—88-92 oil cells/square cm.
Flesh:
   Number of segments.—Average between 9 and 10 segments per fruit.
   Segment walls.—Medium firm with sufficient strength to maintain integrity as separated.
   Juice.—Abundant.
   Color.—Uniformly RHS 23B (yellow-orange).
   Juice color.—42.6 (November).
   Texture.—Medium soft.
   Vesicles.—Length: arranged from 11.0 to 12.5 mm on average Diameter (thickness): 3.5 to 4.2 mm in average.
   Eating quality (varies from season to season).—2012 mid-November data.
   Soluble solids (average).—14.4 Brix.
   Acidity (average).—0.68%.
   Ratio.—21.2.
Seeds: Seedless (seeds rarely found)
Resistance to disease: Alternaria and citrus scab have not been observed in the trees or fruit currently grown. The original trees are growing in a block of trees heavily impacted by HLB (Huanglongbing or citrus greening). One of the original trees has been diagnosed with HLB, with symptoms being observed in the winter months (first observed in February, 2013); however, symptomatic branches died, and the remainder of the tree appears to be recovering. Thus, the overall tolerance to HLB is unknown at this time.

What is claimed is:

1. A new and distinct cultivar of mandarin tree as illustrated and described herein.
